(19) United States
(12) Patent Application Publication
(10) Pub. No.: US 2026/0337060 A1
(43) Pub. Date: Oct. 29, 2026

(54) IN-LINE ILLUMINATION SOURCES WITH UV LEDS

(71) Applicant: Atlantium Technologies Ltd, Beit-Shemesh (IL)

(72) Inventors: Zohar VARDIEL, Or Yehuda (IL); Omer HALIVNI, Jerusalem (IL); Michael KERTSER, Gedera (IL); Ytzhak ROZENBERG, Caesarea (IL); Yariv ABRAMOVICH, Kfar Gibton (IL)

(73) Assignee: Atlantium Technologies Ltd, Beit-Shemesh (IL)

(21) Appl. No.: 19/595,766

(22) PCT Filed: Jan. 1, 2026
(86) PCT No.: PCT/IL2026/050001

§ 371 Date: Jun. 11, 2026

Related U.S. Application Data
(60) Provisional application No. 63/741,267, filed on Jan. 2, 2025.

(51) Int. Cl. C02F 1/32; A61L 2/10; C02F 1/00

(52) U.S. Cl. CPC C02F 1/325; A61L 2/10; C02F 1/008; A61L 2202/11; A61L 2202/14; C02F 2201/3222; C02F 2201/3225; C02F 2201/3227; C02F 2201/3228; C02F 2201/326; C02F 2209/40; C02F 2303/04

(57) ABSTRACT

Illumination sources, water treatment systems and methods are provided, delivering ultraviolet (UV) illumination for water disinfection. Illumination sources comprise UV LEDs (light emitting diodes) attached to a supporting structure that is insertable into the water treatment system and protrudes into a conduit holding the water. The supporting structure with UV LEDs may be similar in dimensions to commercial UV lamps, and used to replace them, enhancing the disinfection performance. The UV LEDs may be arranged to optimize the delivery of illumination to the water in the conduit, and maximize the efficiency of disinfection. Water treatment systems may comprise multiple UV LEDs supporting structures and/or UV lamps, and optimized with respect to all illumination sources. Thermal conductivity may be enhanced and heat removing elements may be used to remove heat from the operating UV LEDs.

### BACKGROUND OF THE INVENTION

### 1. Technical Field

The present invention relates to the field of ultraviolet (UV) water disinfection, and more particularly, to usage of UV LEDs (light emitting diodes) for water disinfection.

### 2. Discussion of Related Art

U.S. Pat. Nos. 7,560,704, 7,683,344, 7,763,177, 8,414,779, 9,320,818, which are incorporated herein by reference in its entirety, disclose various apparatuses and methods for disinfecting water flowing in a conduit using ultraviolet (UV) illumination.

Commercial UV lamps (e.g., MPUV—medium pressure UV lamps and LPUV—low pressure UV lamps) are high-pressure high-temperature mercury or mercury-halide lamps that are used as efficient UV sources for disinfecting water. UV LEDs (light emitting diodes) are semiconductor devices that emit UV at lower intensity but higher power efficiency compared to UV lamps.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides an illumination source for delivering ultraviolet (UV) illumination in a water treatment system, the illumination source comprising a plurality of UV LEDs (light emitting diodes) attached to a supporting structure that is insertable into the water treatment system, protruding into a conduit holding the water.

One aspect of the present invention provides water treatment systems comprising multiple UV LEDs illumination sources and/or at least one MPUV (middle pressure UV) lamp, with the spatial distribution of the UV LEDs on each illumination source being determined to maximize the efficiency of UV delivery, taking into account some or all illumination sources.

One aspect of the present invention provides a method of delivering ultraviolet (UV) illumination for water disinfection, the method comprising: attaching a plurality of UV LEDs (light emitting diodes) to a supporting structure that is insertable and protrudes into a water conduit holding the water, and operating the UV LEDs to disinfect the water.

These, additional, and/or other aspects and/or advantages of the present invention are set forth in the detailed description which follows, possibly inferable from the detailed description, and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. The patent or application file contains at least one drawing executed in color, e.g., FIG. 3A indicating temperature using a color code, FIG. 4B indicating flow velocity using a color code, FIGS. 4C-4E indicating power using a color code and FIGS. 5A and 5B in which color enhances the clarity of the graphs. Copies of this patent or patent application publication with color drawing(s) will be provided by the Office upon request and payment of the necessary fee. In the accompanying drawings:

FIG. 1A is a high-level schematic perspective illustration of a part of an illumination source for delivering ultraviolet (UV) illumination in a water treatment system, according to some embodiments of the invention.

FIG. 1B is a high-level schematic perspective cross section illustration of a part of the illumination source within a transparent casing, which protrudes into a conduit holding water, according to some embodiments of the invention.

FIGS. 2A-2F are high-level schematic illustrations of adjustments of the UV illumination pattern generated by illumination source 100, according to some embodiments of the invention.

FIGS. 3A-3F are high-level schematic illustrations of thermal management configurations of illumination sources, according to some embodiments of the invention.

FIG. 4A is a high-level schematic cross section illustration of a conduit with a transparent casing holding the illumination source within the volume of the conduit with water, according to some embodiments of the invention.

FIGS. 4B-4E provide simulation results for the adjustments of disclosed illumination sources 100 to a given flow pattern, according to some embodiments of the invention.

FIGS. 5A and 5B provide simulation results for estimating the efficiency of disclosed illumination sources in UV disinfection, according to some embodiments of the invention.

FIG. 6 is a high-level flowchart illustrating a method of delivering ultraviolet (UV) illumination for water disinfection, according to some embodiments of the invention.

FIG. 7 is a high-level block diagram of a non-limiting example of a controller, which may be used with embodiments of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Some embodiments of the present invention provide efficient and economical methods and mechanisms for using UV LEDs for water disinfection and thereby provide improvements to the technological field of UV water disinfection. Illumination sources, water treatment systems and methods are provided, delivering ultraviolet (UV) illumination for water disinfection. Illumination sources comprise UV LEDs (light emitting diodes) attached to a supporting structure that is insertable into the water treatment system and protrudes into a conduit holding the water (as a non-limiting example for any disinfection reactor, typically handling flowing water or other liquids). Thermal conductivity may be enhanced and heat removing elements may be used to remove heat from the operating UV LEDs. The supporting structure with UV LEDs may be similar in dimensions to commercial MPUV (middle pressure UV) lamps, and used to replace them, enhancing the disinfection performance. The UV LEDs may be arranged to optimize the delivery of illumination to the water in the conduit, and maximize the efficiency of disinfection. Water treatment systems may comprise multiple UV LEDs structures and/or UV lamps, and optimized with respect to all illumination sources.

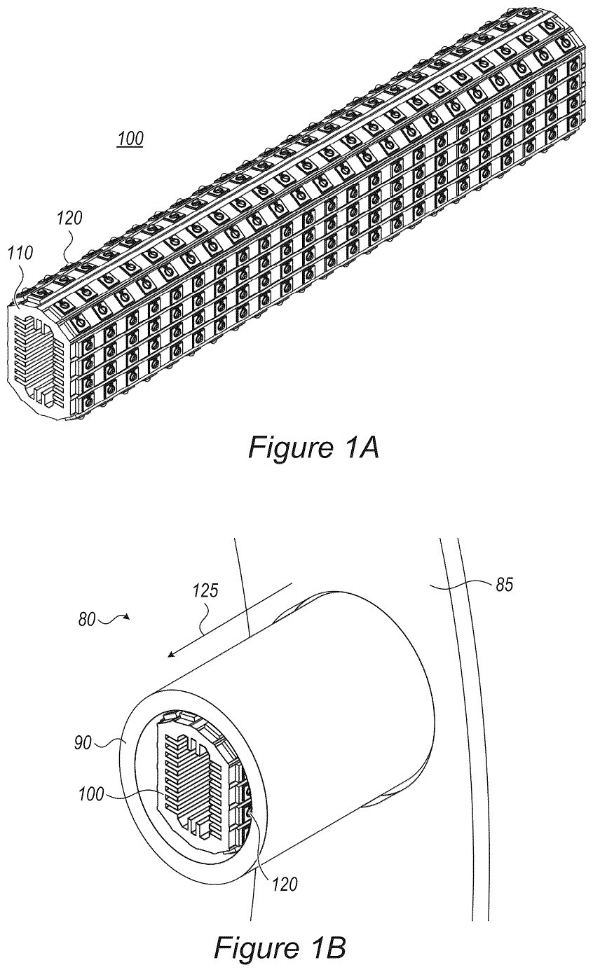

FIG. 1A is a high-level schematic perspective illustration of a part of an illumination source 100 for delivering ultraviolet (UV) illumination in a water treatment system, according to some embodiments of the invention. FIG. 1B is a high-level schematic perspective cross section illustration of a part of illumination source 100 within a transparent casing 90, which protrude into a conduit 85 holding water 80, according to some embodiments of the invention.

Illumination source 100 comprises a plurality of UV LEDs (light emitting diodes) 120 attached to a supporting structure 110 that is optionally insertable into UV-transparent casing 90 of the water treatment system, with UV-transparent casing 90 and illumination source 100 protruding into conduit 85 holding the water (typically water flowing through the conduit). It is noted that in various embodiments, illumination source(s) 100 may be configured to be directly insertable into the water treatment system and protrude into conduit 85 holding the water—with corresponding electrical insulation of UV LEDs 120 from the water. In case of simple replacement of a commercial UV lamp by disclosed illumination source 100, configured to have the same shape or a compatible shape as the commercial UV lamp, illumination source 100 may be configured to be insertable into UV-transparent casing 90 for housing the commercial UV lamp. However, UV-transparent casing 90 is not required for disclosed UV LED-based illumination source 100 as it is for commercial UV lamps, and hence may be removed when retrofitting a given system.

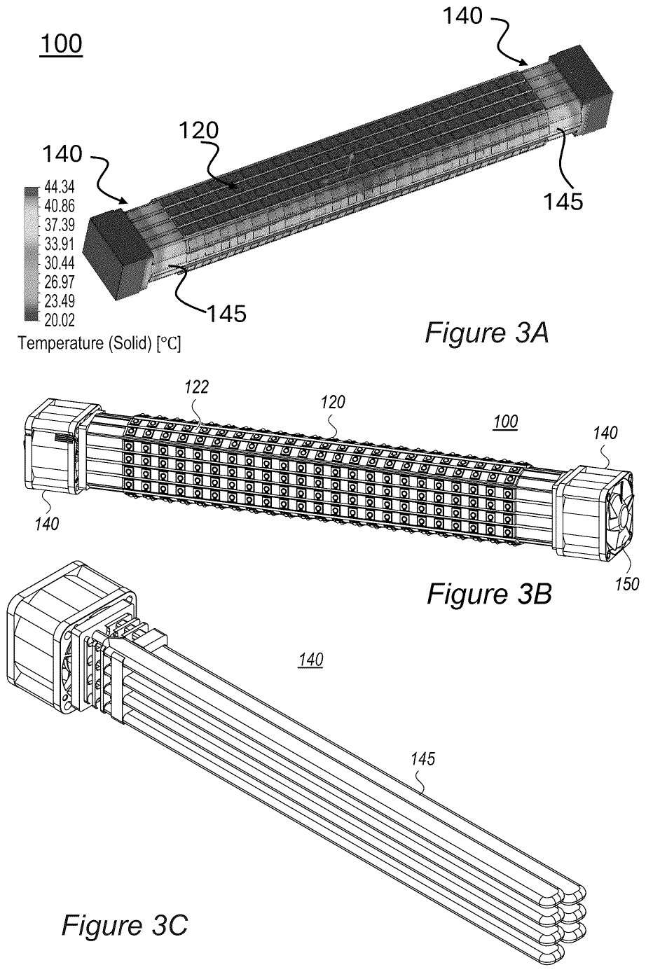

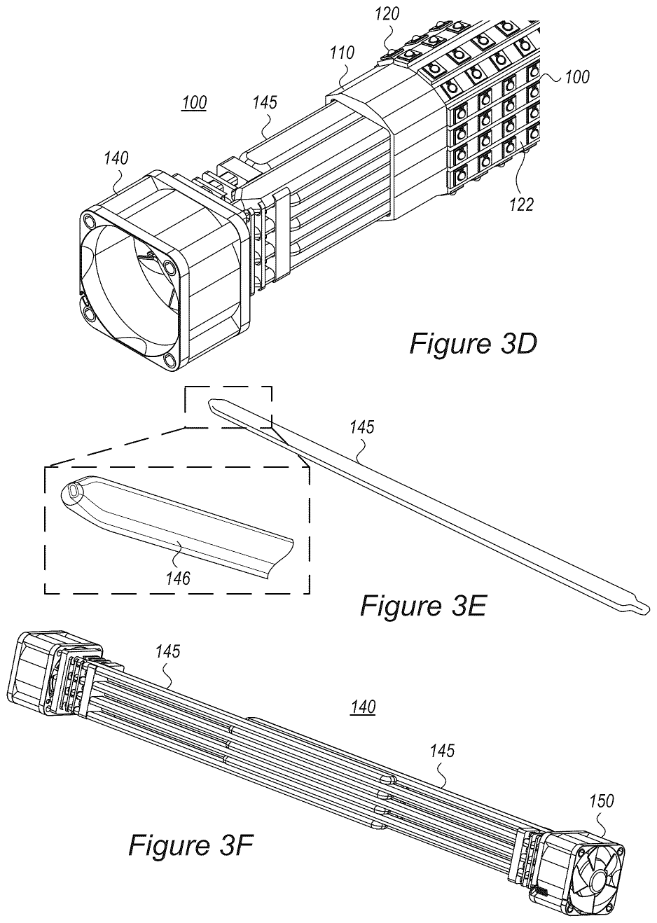

Structure 110 that supports LEDs 120 may comprise a heat-conductive body (for example, made of aluminum or copper) designed to accommodate modular MCPCB (metal core printed circuit board) strips 122 (see FIGS. 3B and 3D) with LED sources 120 attached to the MCPCB. The strips may comprise multi-layer PCB (printed circuit board) structures, specially fit to provide electrical connection to LEDs 120 with overcurrent protection and redundancy mechanism to accommodate single LED failure without further effects on illumination source 100. In various embodiments, the density, type and mutual orientation of LEDs 120 over the strip and around supporting structure 110 may be varied with respect to the geometry of the disinfection reactor (represented herein in a schematic manner as conduit 85) and with respect to specific application requirements.

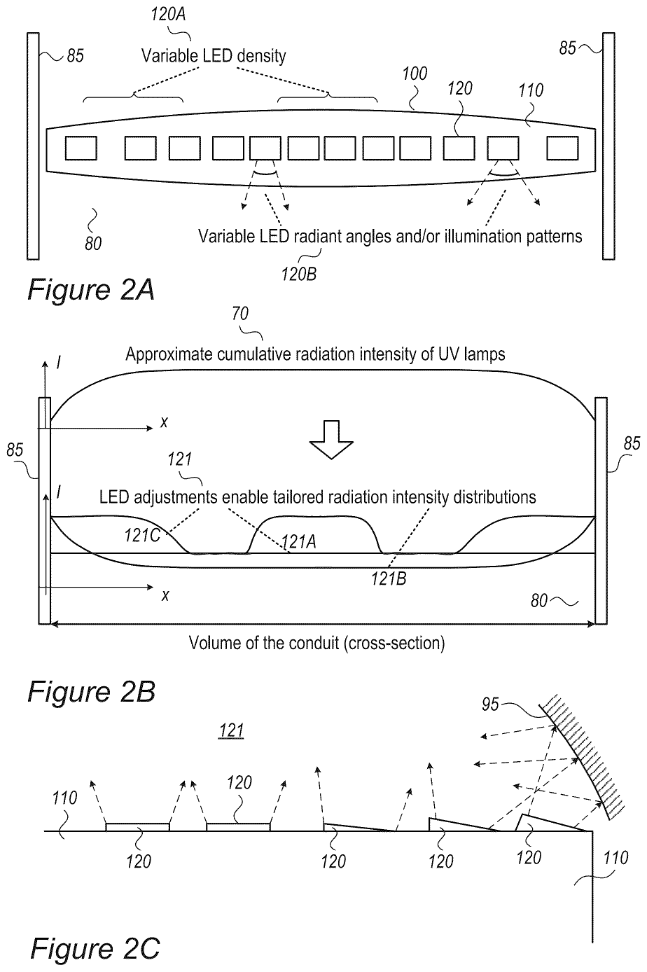

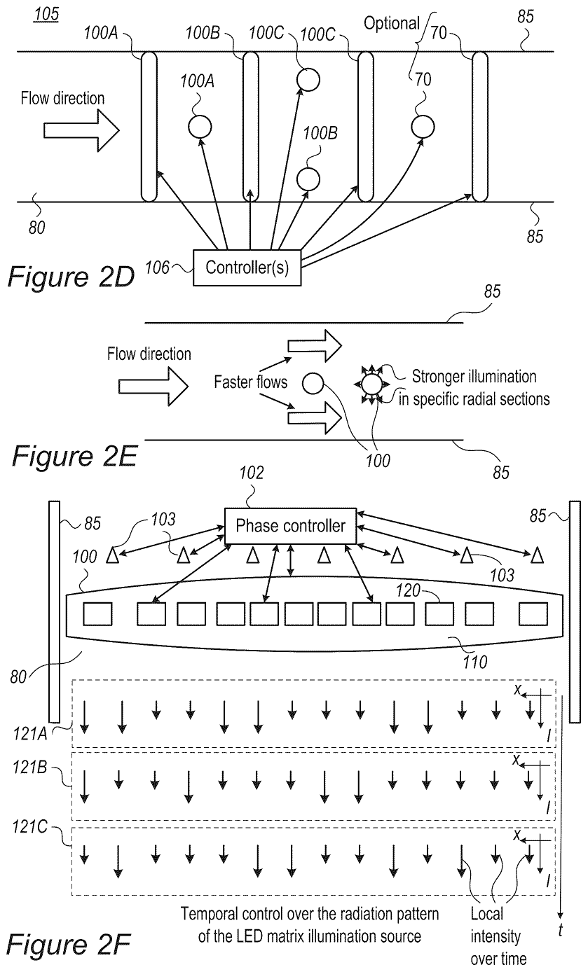

In various embodiments, LEDs 120 may comprise units that illuminate at 265 nm with typical optical power of 100 mW each. In other embodiments, different LEDs 120 may be used, e.g., 255 nm LEDs, LEDs within the VUV-range (100 nm to 300 nm), UV-A LEDs (within the range 315 nm to 400 nm) as well as mixture LEDs 120 at different wavelengths along the PCB strip and/or around supporting structure 110 (illumination source 100). In various embodiments, the power of LEDs 120 may also vary, with respect to the application requirements and technological limitations. Individual LED optics may be used to adjust the radiation pattern of individual LEDs (see, e.g., FIGS. 2A-2D) and/or phase-controlled operation over the LED matrix may be used to manipulate the entire source radiation pattern (see, e.g., FIG. 2F).

Advantageously, using specific single wavelength spectra of UV LEDs 120 may provide better disinfection results than commercial polychromatic UV lamps (e.g., MPUV or LPUV lamps) due to selectivity of radiant properties. Moreover, using MCPCB strips that connect LEDs 120 may also be utilized to conduct heat to supporting structure 110, e.g., to dissipate the heat outside the system (rather than in the water in the conduit as in commercial MPUV lamp applications), for example using thermal-conductive heat-pipes.

In various embodiments, structure 110 may have a shape of a commercial MPUV (medium pressure UV) lamp, so that UV lamps (e.g., MPUV lamps) in operating or planned water treatment systems of various types may be replaced (retrofitted) with disclosed illumination source 100 without changing their mechanical design, improving performance with minimal required adjustments to the systems. In some embodiments, the disinfection efficiency of illumination source 100 is higher than a disinfection efficiency of a commercial MPUV lamp having similar dimensions and illumination power.

Supporting structure 110 with the UV LEDs 120 may be pulled out, e.g., for maintenance, as a single unit. The similar structural and mechanical configuration allows for simple upgrades of existing MPUV reactors into disclosed reactors, by replacing one, some or all the UV lamps with disclosed illumination source 100, without requiring, or requiring minimal structural adjustments of the disinfection systems as a whole.

FIGS. 2A-2F are high-level schematic illustrations of adjustments of the UV illumination pattern generated by illumination source 100, according to some embodiments of the invention.

In some embodiments, UV LEDs 120 may be arranged on structure 110 to maximize an efficiency of UV delivery into water 80 in conduit 85. For example, FIG. 2A schematically illustrates a non-limiting example of a matrix of UV LEDs 120 arranged with a changing, or variable LED density 120A along the axis of structure 110, e.g., across the diameter of conduit 85. Adjusting the distribution and density (120A) of UV LEDs 120 may be carried out to concentrate more radiation in areas of high fluid velocity (e.g., locally higher throughput of water 80) and less radiation in the areas of low flow velocities, to make a UV dose distribution narrower, and thus more energy-efficient.

In some embodiments, UV LEDs 120 may be arranged on supporting structure 110 to provide a specified pattern of illumination. For example, UV LEDs 120 may be arranged to deliver more light further away from the sides of the conduit and into the flow regions. For example, in some embodiments, a number of UV LEDs 120 may increase from a proximal end of structure 110 adjacent to walls of conduit 85—toward a distal end of structure 110 away from the walls of conduit 85 and towards a center of conduit 85 (illustrated schematically in FIG. 1B by arrow 125).

FIG. 2A also illustrates schematically a non-limiting example of configuring the radiant patterns of individual UV LEDs 120 to vary along supporting structure 110, in relation to functional and/or operational considerations—according to specified changing, or variable LED radiant angles and/or illumination patterns 120B, indicated schematically. For example, radiant optical angles of individual LEDs 120 may range, e.g., between 10° to 120°, or have any intermediate values, e.g., 30°, 50°, 70°, 90°, 100°, etc., angle indicated schematically in FIG. 2A) in order to adjust the optical radiant patterns to the application needs, e.g., in relation to the UV-transparency of water, the reactor geometry (e.g., conduit 85 may have regions with different diameters, different cross-sectional shapes, bifurcations, etc.). Advantageously, the capability to adjust the radiation patterns is advantageous with respect to UV lamps in that the radiant pattern of the latter is fixed, more or less even along the axis, with some intensity drop around the edges.

FIG. 2B illustrates in a highly schematical manner the adjustable radiation intensity distributions 121 by disclosed illumination source 100, which may be tailored as required by adjusting the positions and radiation patterns of LEDs 120 along structure 110, compared to prior art fixed, approximate radiation intensity of UV lamps 70. While the latter is fixed, typically having lower illumination intensity at the edges of conduit 85 (indicated schematically, and with I and x axes denoting the intensity and spatial coordinates), disclosed LED configurations may be tailored as explained in FIG. 2A to provide pre-defined distributions of illumination intensity across conduit 85, exemplified in a non-limiting manner by three schematic distributions—a uniform intensity distribution 121A, which is not achievable using prior art lamps, a density distribution 121B with enhanced illumination intensity at the edges of conduit 85 (contrasting with prior art illumination intensity distribution 70) and a fluctuating illumination intensity distribution 121C, which may be useful under specific flow regimes through conduit 85. Clearly any intermediate or different intensity distribution may be provided, as disclosed herein.

The insertion of illumination source 100 within conduit 85 (as a non-limiting example for disinfection reactors) enables to provide a great variety of radiation patterns to disinfect the flowing liquid as required, in contrast to LED applications with LEDs set only on the outside of conduit 85—a configuration which is much more limited with respect to the possibilities of radiation intensity profile design of disclosed illumination sources 100.

FIG. 2C illustrates further adjustments in the configuration of LEDs 120 along and around supporting structure 110 in association with reflective elements(s) 95 placed on the walls or within conduit 85 (not illustrated in FIG. 2C). These enable an even higher diversity of illumination intensity patterns 121 (indicated schematically by the dashed arrows) in achieving specified illumination intensity profiles with respect to a wide range of conduit and flow geometries, and placement of illumination source 100 with respect thereto. In some embodiments, the water treatment system may comprise at least one reflective element 95, positioned, e.g., on the walls of the reactor (conduit 85), inside the reaction chamber (conduit 85) and/or possibly on structure 110 itself. UV LEDs 120 may be arranged to maximize the efficiency of UV delivery, taking into account angular considerations relating to a position and orientation of reflective element(s) 95. In a non-limiting example, FIG. 2C illustrated schematically a curved mirror as reflective element 95 and adjustments of the angles and possibly positions at which UV LEDs 120 are set on structure 110, to take into account the reflection of the radiation omitted by UV LEDs 120. It is noted that setting LEDs 120 at different angles provides additional flexibility in adjusting illumination intensity pattern 121 to pre-defined performance requirements.

In some embodiments, reflective element 95 may comprise a back surface (e.g., coated with a reflective coating) of conduit 85 or other element(s) in water treatment system 105 (see general schematic illustration in FIG. 2D). The distribution of UV LEDs 120 may be optimized with respect to the flow of water in conduit 85 and the presence and location of reflective element 95 in the water treatment system.

Some embodiments comprise a water treatment system comprising at least one UV lamp (MPUV and/or LPUV lamps) and at least one illumination source 100, wherein a spatial distribution of UV LEDs 120 of illumination source(s) 100 is determined to maximize the efficiency of UV delivery, taking into account an illumination distribution of the at least one UV lamp. For example, fewer LEDs may be positioned adjacent to or in the direction of the UV lamp(s), and more LEDs may be directed to regions that are not covered, or covered to a less extent by the UV lamp(s).

Some embodiments comprise a water treatment system 105 comprising multiple illumination sources 100, wherein a spatial distribution of UV LEDs 120 on each illumination source 100 is determined to maximize the efficiency of UV delivery, taking into account relative positions of illumination sources 100.

FIG. 2D provides a schematic illustration demonstrating water treatment system 105 with multiple illumination sources, some of which may comprise disclosed illumination sources 100A, 100B, 100C with similar or different configurations of UV LEDs 120 on respective structure 110, while others may comprise commercial UV lamps (e.g., MPUV and/or LPUV lamps) 70. Water treatment system 105 also demonstrated schematically how UV lamp(s) in commercial reactors may be replaced (retrofitted) with disclosed illumination sources 100, as supporting structures 110 are configured to be mechanically compatible with UV lamps.

In the schematic non-limiting illustration, different illumination sources 100 may be positioned with different orientations and possibly with different configurations of UV LEDs 120 on respective structures 110—provide specified illumination patterns to optimize disinfection, e.g., under different performance requirements, fluid characteristics, flow regimes, etc. LED density and illumination pattern along each illumination source 100 may be adjusted with respect to nearby UV sources and overall radiation requirements, indicated schematically as illumination sources 100A, 100B, 100C with potentially different illumination intensity patterns 121, achieved, e.g., by different LED parameters and distribution, different placement angles of LEDs, different reflective elements, etc., as illustrated schematically in FIGS. 2A-2C. For example, the density, distribution, optical pattern and relation of reflective elements of individual UV LEDs 120 may be adjusted respectively, as described herein. In some embodiments, the optical characteristics of disclosed illumination sources 100 may be adjusted during operation, e.g., with respect to changing flow or fluid parameters, for example by a controller 106 as part of water treatment system 105. Controller 106 may be implemented as controller 60 (see FIG. 7).

FIG. 2E illustrates schematically radial variation in illumination intensity, according to some embodiments of the invention. In some embodiments, the distribution and/or operation of LEDs 120 may vary radially, with respect to the axis of illumination source 100. For example, more LEDs may be used to illuminate sections of the flow through conduit 85 which are characterized by higher flow speeds or higher opacity (possibly measured by sensors 86, 103, and/or according to the system's design), which allow for a shorter distance for effective illumination—in order to ensure the application of sufficient UV dose to these regions (illustrated in a highly schematic manner).

Alternatively or complementarily, the operation pattern of the respective LEDs may vary with respect to spatio-temporal characteristics (or changes) of flow patterns in conduit 85. LEDs 120 may be around illumination source 100 in a pattern that is adjusted to the expected flow profile in the conduit (e.g., denser facing regions with higher flow speeds or higher opacity and sparser facing regions with lower flow speeds or lower opacity) in a predefined profile which is not radially symmetric. In some embodiments, operation of LEDs 120 may be carried out to further enhance the radially asymmetric illumination pattern. Illumination control may be implemented in various ways, such as controlling the current provided to each LED or groups of LEDs and controlling the intensity of illumination with respect to spatio-temporal changes of flow patterns and opacity/visibility in conduit 85, or switching individual LEDs or groups of LEDs on and off according to the required illumination intensity at different sections (e.g., radial sections) of illumination source 100, possibly with respect to reflective elements 95.

In various embodiments, the number of UV LEDs 120 and/or an operation intensity thereof increases at radial sections characterized by shorter effective illumination distances. The modification of the illumination intensity may be carried out by adjustment of the currents delivered to UV LEDs 120 and/or by switching UV LEDs 120 to operation for a duration proportional to the required illumination intensity.

FIG. 2F illustrates schematically a configuration of a matrix of LEDs 120 on supporting structure 110, which is controlled by a phase controller 102 to provide phase-controlled, temporally-adjusted operation over the LED matrix, adapting radiation pattern 121 of illumination source 100 (indicated schematically by consecutive patterns 121A, 121B, 121C) to given flow patterns in conduit 85, which may change over time for various reasons. In addition to changes in the overall flow throughput, to which the intensity of individual UV LEDs 120 may be adjusted, also local changes in flow speed and direction may occur, e.g., due to turbulence or other non-linear phenomena (e.g., forming of vortices in the flow, e.g., Kármán vortex street)—to which illumination intensity profile 121 may also be adjusted. Phase control may be implemented in various ways, such as controlling the current provided to each LED or groups of LEDs and controlling the intensity of illumination with respect to spatio-temporal changes of flow patterns in conduit 85, or switching individual LEDs or groups of LEDs on and off according to the required illumination intensity at different sections of illumination source 100, possibly with respect to reflective elements 95.

In various embodiments, phase controller 102 may be configured to set phased driving currents over the LED matrix (controlled with respect to individual or groups of LEDs 120) to provide a radiation pattern that is tightly fitted to the selected reactor geometry or flow pattern. For example, phase controller 102 may be configured to drive UV LEDs 120 across the surface of illumination source 100 to provide more power at the center of illumination source 100 (operating the central LEDs at a higher intensity than the peripheral LEDs) to provide higher illumination power at high flow regions and less power to slower flow regions at the edges of conduit 85.

In case of variable, non-stationary flow, possibly having a periodic flow pattern (e.g., Kármán vortex street) phase controller 102 may be configured to drive UV LEDs 120 to adjust the power of UV LEDs 120 at the edges of illumination source 100 to fit the radiation intensity to the temporal pattern of detachment of the formed eddies. In some embodiments, illumination source 100 may be controlled in a feedback loop, modifying illumination patterns with respect to measured flow patterns. For example, mass-flow sensors 103 may set into conduit 85 and configured to monitor the flow of fluid 80 (e.g., water) inside the reactor volume (conduit 85) and phase controller 102 may be configured to adjust the illumination intensity of individual UV LEDs 120 or groups of UV LEDs 120 with respective to the sensor readings to yield corresponding illumination patterns 121.

FIG. 2F illustrates in a highly schematical manner a set of mass-flow sensors 103 within water 80 flowing through conduit 85 (e.g., mass-flow sensors 103 may be attached to walls of conduit 85 to the surface of illumination source 100 and/or to other elements within the reactor), configured to measure the temporal changes in flow and provide the measurements to phase controller 102 (e.g., by wire or wireless). Phase controller 102 may respectively adjust the illumination intensity of UV LEDs 120 to adjust illumination pattern 121 of illumination source 100 to transient changes in the flow pattern—indicated in FIG. 2F in a highly schematic manner by the small arrows at the bottom of the figure, suggesting different patterns 121A, 121B, 121C of illumination intensities at different times. Phase controller 102 may be implemented as controller 60 (see FIG. 7). Disclosed embodiments thus provide temporal control over radiation pattern 121 of LED matrix illumination source 100 by adjusting the local illumination intensity over time and throughout the treated volume in conduit 85.

Advantageously, illumination sources 100 provide the delivery of UV radiation within the internal volume of conduits 85, in contrast to prior art applications that employ UV LED matrices placed outside conduits 85, e.g., on transparent portions of their external walls. Placing illumination source(s) 100 within the volume of conduits 85 (directly, or within UV-transparent casing(s) 90, e.g., made of quartz) improves the efficiency of UV delivery, increases its effectiveness in disinfecting the water and provides much more flexibility in treating different volumetric portions of the conduit's interior. Advantageously compared to UV lamps that have a fixed spatial illumination distribution, disclosed illumination sources 100 may be optimized to provide specified spatial illumination distribution tailored to the application, shape of conduit and water flow pattern therethrough, and under consideration of other illumination sources used to disinfect the water flowing through the conduit.

FIGS. 3A-3F are high-level schematic illustrations of thermal management configurations of illumination source 100, according to some embodiments of the invention. In some embodiments, illumination sources 100 may comprise heat dispersal elements 140 configured to remove excessive heat produced by densely packed UV LEDs 120 during their operation. For example, heat dispersal elements 140 may comprise heat conveying pipes 145 positioned within supporting structure 110 that may be configured as a sleeve, and/or heat removing elements 150 such as ventilators and/or radiators configured to remove heat from heat conveying pipes 145. Heat dispersal elements 140 may comprise additional or complementary heat management solutions, such as thermal paste (comprising thermally conductive materials) enabling or improving heat transfer away from UV LEDs 120 (e.g., via MCPCB strips 122), heat monitoring controller(s), heat exchangers, etc. to keep UV LEDs 120 at operational temperatures and avoid heating the water in conduit 85 beyond specified limits. Various configurations of heat dispersal elements 140 and illumination source 100 may be used to provide close contact and efficient heat transfer from UV LEDs 120 through heat dispersal elements 140 and away from illumination source 100.

FIG. 3A illustrates simulation results for heat generation by UV LEDs 120 and heat removal by heat dispersal elements 140 including heat conveying pipes 145, which are shown in the illustration to be cooler and therefore remove heat from UV LEDs 120. FIGS. 3B-3F further illustrate the structure of heat dispersal elements 140 within illumination source 100, which may include two opposing sets of heat conveying pipes 145, mechanically interspaced and fitting together to form efficient heat removal from sleeve-shaped supporting structure 110 with UV LEDs 120. For example, FIG. 3D illustrates schematically one of heat dispersal elements 140, FIG. 3F illustrates schematically a side-by-side view of heat dispersal elements 140 illustrating their interspacing (e.g., heat conveying pipes 145 of one heat dispersal elements 140 being on one side and heat conveying pipes 145 of the other heat dispersal elements 140 being on the other side), and FIG. 3C illustrates the fitting together of heat conveying pipes 145 of both opposite heat dispersal elements 140 to form an effective heat conveying structure (somewhat pulled out of supporting structure 110 sleeve to illustrate the structure). FIG. 3B illustrates operable illumination source 100, with heat removing elements 150 as ventilators on one or both sides of illumination source 100 to disperse the removed heat. FIG. 3E illustrates a non-limiting example for a structural configuration of heat conveying pipes 145, which may comprise a slit along 146 the length of heat conveying pipes 145, e.g., implemented by a longitudinally folded thin copper foil or sheet.

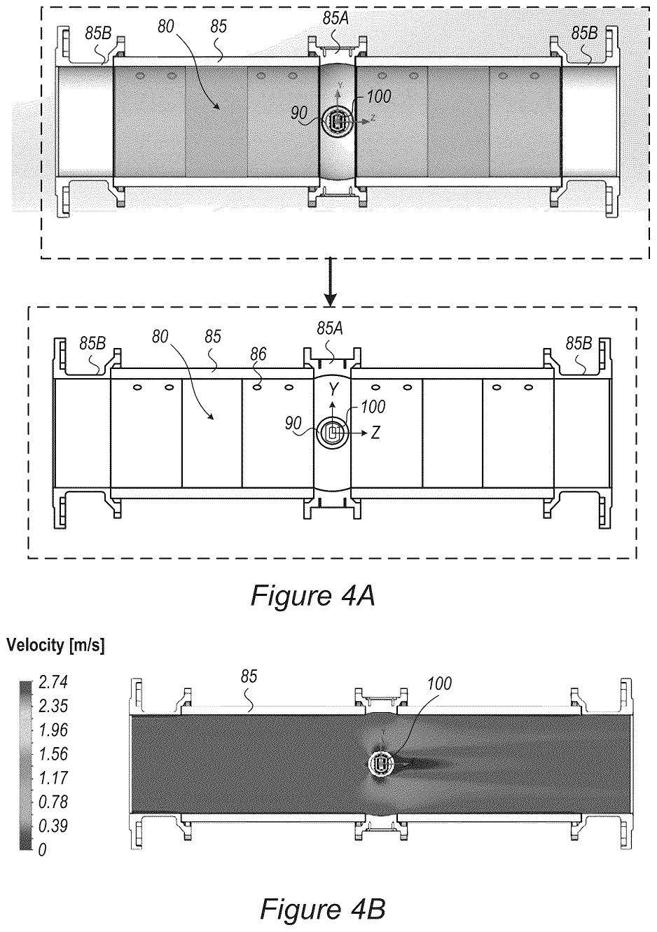

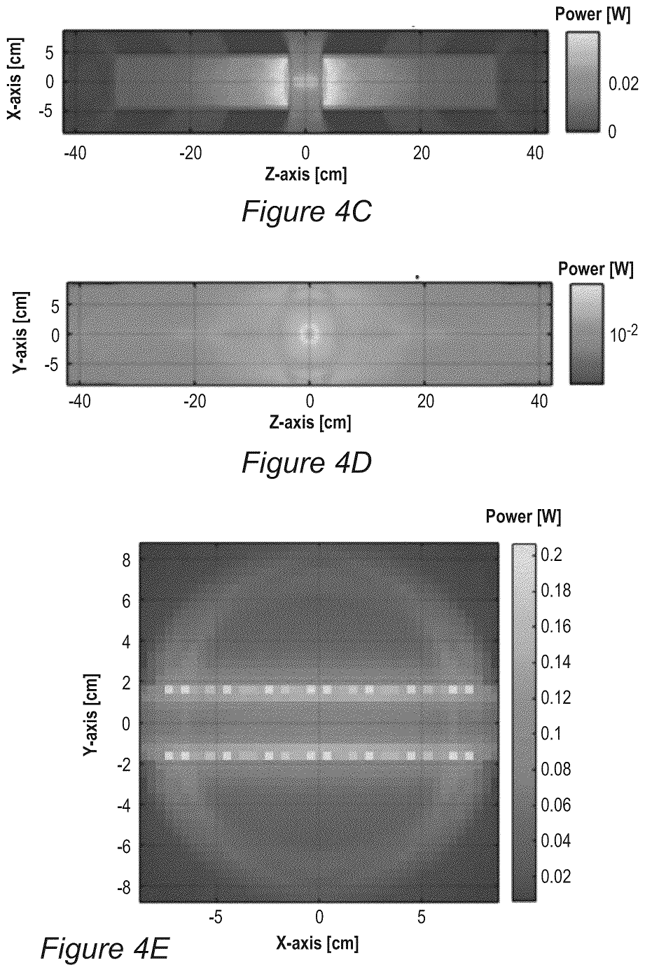

FIG. 4A is a high-level schematic cross section illustration of conduit 85 with transparent casing 90 holding illumination source 100 within the volume of conduit 85 with water 80, according to some embodiments of the invention. FIG. 4A includes an image and a corresponding schematic illustration of the experimental setting for the results provided in FIGS. 4B-4E, with a central section 85A in which transparent casing 90 and illumination source 100 are installed, and connectors 85B for attaching conduit 85 to further elements such as pumps and pipework. Z axis is denoted along conduit 85, X axis is defined along illumination source 100 and Y axis is denoted across conduit 85 and perpendicularly to illumination source 100. In various embodiments, sensors 86 (e.g., mass-flow sensors 103 disclosed herein) may be set at different positions along conduit 85 to provide flow measurements and possibly additional measurements of the liquid—which may be used by any of the disclosed controllers to adjust any of the parameters of the illumination.

FIGS. 4B-4E provide simulation results for the adjustments of disclosed illumination sources 100 to a given flow pattern, according to some embodiments of the invention. FIG. 4B represents the flow velocity distribution of fluid 80 within conduit 85 over the cut-plot in the Y-Z plane. The flow is shown to have a high velocity over the top and the bottom of sleeve 90 (surrounding illumination source 100) and stagnation (low velocity) after sleeve 90. The density and the spatial intensity of LEDs 120 may be adjusted with respect to this flow pattern, as disclosed herein.

FIGS. 4C-4E illustrate schematically the adjustment of illumination density to the flow pattern illustrated in FIG. 4B, providing volumetric flux cut-plots in the X-Z plane (longitudinal cross section), Z-Y plane (longitudinal cross section) and X-Y plane (transverse cross section, with individual LEDs visible), respectively, indicating the illumination intensity (expressed as volumetric power flux, V/V to represent the simulated intensity) that UV LEDs 120 are configured and arranged to deliver, to optimally handle the flow pattern.

For example, in some embodiments, the supporting structure may comprise 16 lines of 23 LEDs each (totaling 368 UV LEDs), with each UV LED having an optical power of 200 mW at 265 nm—reaching 73.6 W an optical power, which is similar to a commercial MPUV lamp; however with electrical power consumption of 837.2 W (ca. 9% WPE, wall plug efficiency). UV LEDs 120 may be arranged symmetrically around an axis of structure 110 and along a length of structure 110, as illustrated schematically in FIG. 1A.

Various electric connections may be applied between UV LEDs 120, e.g., each line of UV LEDs 120 may be connected in series and the lines may be connected in parallel. For example, in various embodiments, each MCPCB LED strip 122 may comprise one or more groups of UV LEDs 120 in series. Each group of UV LEDs 120 may comprise between 1 and 50 UV LEDs 120 (possibly between 2-5, 2-10, 5-20, 10-30, 20-50 or any intermediate value) and a dedicated constant current driver(s) (not shown) configured to limit the current supplied to the respective group of UV LEDs 120. Illumination source 100 may comprise between 1 and 100 groups of UV LEDs 120 (possibly between 2-15, 5-20, 10-30, 20-50, 50-100 or any intermediate value) connected in parallel, in series or in a combination of serial and parallel connections. In total, illumination source 100 may be configured to comprise between 5 and 200 UV LEDs 120 per MCPCB LED strip 122, and may include multiple MCPCB LED strips 122—in various groupings and forms of electrical connections, according to various specifications.

The supply voltage for each MCPCB strip may be between 5-500V, depending on the number of LEDs in each group. The voltage to the MCPCB strips may be supplied through dedicated connectors on one or both sides of the respective MCPCB strips. Alternatively, the voltage may be supplied through the heat sink (e.g., heat dispersal elements 140 or components thereof) and/or the connections (e.g., screws) that connect MCPCB strips 122 to heat dispersal elements 140 or components thereof. In some embodiments, illumination source 100 may comprise 1-50 MCPCB strips 122 that are connected in parallel, in series or in a combination of serial and parallel connections—to a voltage supply in the range of between 5-500V (or possibly between 8-350V or intermediate ranges) and supplied by an external LED driver (not shown). The external LED driver may be configured to reduce the power to the LED strips by using PWM (pulse-width modulation) or current limiting. The external LED driver may be configured to receive operating and control commands from UV system control cards (implementing one or more controllers 60). Current and voltage protection components, such as NTC (negative temperature coefficient) thermistors, PTC (positive temperature coefficient) thermistors, TVS (transient voltage suppression) diodes, etc., may be added to the LED strip supply line.

In non-limiting examples, the configuration of UV LEDs 120 may comprise 3-5 UV LEDs per line, arranged in 5-8 groups connected in parallel per MCPCB strip 122, with 16 such MCPCB strips 122 connected in parallel to form illumination source 100 resulting in in 400-384 UV LEDs 120 operating under 40-24V, respectively. In various configurations, the number of UV LEDs per line may be changed (e.g., within 1-50 or intermediate values), the number of groups may be changed (e.g., within 1-50 or intermediate values), the number of MCPCB strips may be changed (e.g., within 1-50 or intermediate values), and the connection pattern and operation voltage may be changed as adjusted according to various specifications.

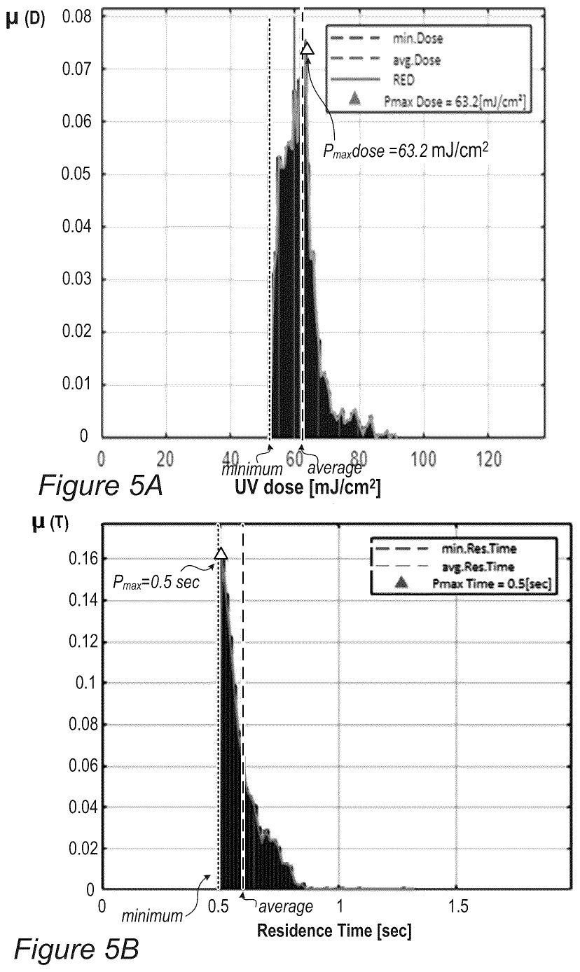

FIGS. 5A and 5B provide simulation results for estimating the efficiency of disclosed illumination source 100 in UV disinfection, according to some embodiments of the invention. Table 1 summarizes the results and assumptions of the simulations, as further explained below.

The following terms and expressions are used: The Residence time (T) denotes the time a given parcel of water spends inside the UV-illuminated region of the conduit; μ(T) denotes the probability density function (PDF) of the residence time (T) across all simulated fluid trajectories; and μ(D) is the probability density function of the delivered UV dose across all water parcels. FIGS. 5A and 5B illustrate the probability density distributions of the ultraviolet dose μ(D) and of the residence time μ(T), respectively, computed over simulated fluid trajectories through the illuminated region of the conduit. The residence time represents the duration for which individual fluid parcels remain within the UV exposure volume, while the UV dose represents the time-integrated ultraviolet intensity experienced along each trajectory. The resulting distributions demonstrate a narrow spread with respect to both residence time and delivered dose, indicating uniform hydraulic transport and effective coupling between flow patterns and ultraviolet illumination.

TABLE 1
Results and assumptions of the simulations.

| | Explanations | Comments |
|---|---|---|
| Results | | |
| RED value = 59.8 | RED—Reduction | |
| mJ/cm² | equivalent dose, the | |
| | effective UV dose. | |
| Minimum dose = 51.8 | | |
| mJ/cm² | | |
| Average dose = 62.0 | | |
| mJ/cm² | | |
| TUF = 0.97 | TUF—Trajectory | An internal metric, |
| | uniformity factor, | optimal at 1. |
| | defines how well | Better than for |
| | the system | commercial MPUV |
| | geometry and flow | lamps. |
| | pattern are | |
| | coupled with the | |
| | optics. | |
| DSL value = 0.16 | Proximity of the | An internal metric, |
| | minimum dose to | the smaller the more |
| | the average dose. | uniform the distri- |
| | | bution of dose. Better |
| | | than for commercial |
| | | MPUV lamps. |
| Assumptions | | |
| D-1Log = 18.0 mJ/cm² | The UV dose required | |
| | to reduce the pathogen | |
| | concentration by 1 log | |
| Etta_g = 1 | η_{g}—the percentage of | Germicidal efficiency, |
| | optical power that is | UV LEDs can be |
| | efficiently converted to | tuned to approach |
| | germicidal power. | η_{g} = 1 |

The results were calculated for D−1 Log (a 1-log UV dose, required to reduce the pathogen concentration by 1 log)=18.0 [mJ/cm²] and Etta_g (η_{g}, germicidal efficiency−the percentage of optical power that is efficiently converted to germicidal power)=1.000. It is noted that Etta_g=1 is very generous assumption, but it is expected to be close for UV LEDs, which are monochromatic and can be selected to emit UV at DNA absorption peak(s).

The simulated results include the RED (Reduction Equivalent Dose) value (a calculated Equivalent Dose, a value used in the industry, based on 1-log inactivation and dependent on flow and UV-transparency of water)=59.8 [mJ/cm²](compared with RED value for a representative commercial MPUV lamp system operating with 1.7 kW electrical power at 100 m³/h flow, 98%-1 cm UVT of 124.7 mJ/cm²), the Minimum Dose=51.8 [mJ/cm²], the Average Dose=62.0 [mJ/cm²], the TUF (trajectory uniformity factor, an internal metric which defines how well the system geometry and flow pattern are coupled with the optics, the closer the TUF is to 1, the better)=0.97, and the DSL value (an internal metric which defines a measure of how much the minimum dose is close to average. The smaller the DSL value is, the better, representation a more uniform distribution of dose)=0.16 (it is noted that 0.16 is considered a very good value for a UV-system). It is noted that the simulation results are based on a simplified calculation with the given assumptions, and that the actual configuration of illumination source 100 and UV LEDs 120 thereof depend on additional characteristics and properties which may be calculated at higher accuracy and more detailed simulations and/or measurements.

Compared to commercial MPUV systems, the resulting DSL and TUF (internal metrics) values are very good, and the dose delivery efficiency for water treatment system 105 with illumination sources 100 was found to be 0.071 [W*mJ/cm²] which is quite similar to the dose delivery efficiency of 0.073 [W*mJ/cm²] from commercial MPUV systems. Still, the dose delivery efficiency for water treatment system 105 with illumination sources 100 may be further improved by implementing disclosed adjustments of LED arrangement over supporting structure 110. Moreover, while UV lamp applications require a UV transparent sleeve to isolate UV lamp from the fluid flow in the reactor (e.g., water 80 flowing in conduit 85), in-water wireless LEDs 120 may not require a sleeve, and therefore further enhances the improvement in efficiency.

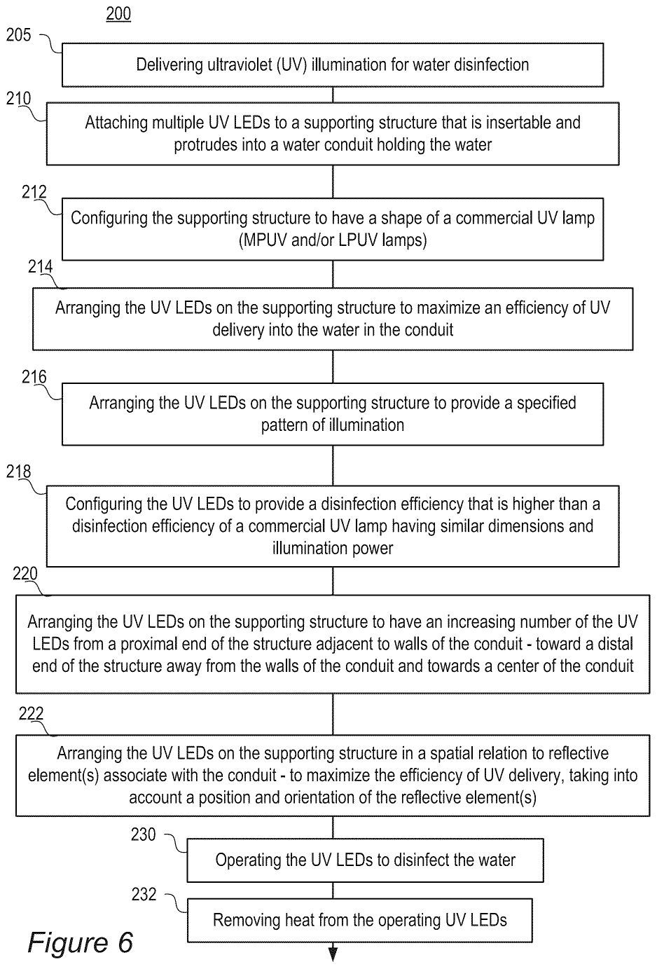

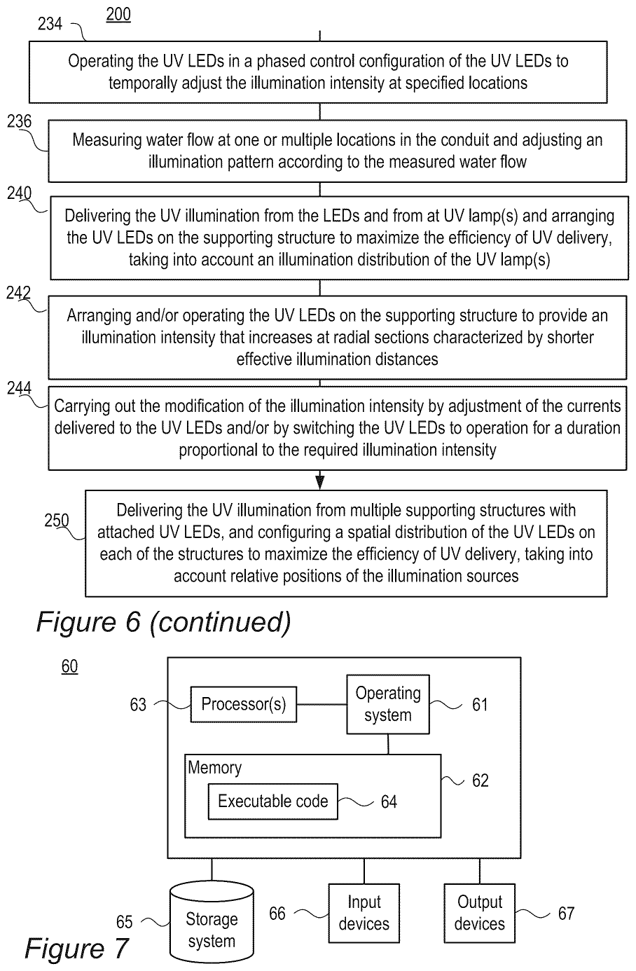

FIG. 6 is a high-level flowchart illustrating a method 200 of delivering ultraviolet (UV) illumination for water disinfection (stage 205), according to some embodiments of the invention. The method stages may be carried out with respect to illumination system 100 described above, which may optionally be configured to implement method 200. Method 200 may comprise the following stages, irrespective of their order.

Method 200 may comprise attaching a plurality of UV LEDs (light emitting diodes) to a supporting structure that is insertable and protrudes into a water conduit holding the water (stage 210), and operating the UV LEDs to disinfect the water (stage 230). Method 200 may further comprise configuring the supporting structure to have a shape of a commercial UV lamp (MPUV and/or LPUV lamps) (stage 212).

Method 200 may further comprise arranging the UV LEDs on the supporting structure to maximize an efficiency of UV delivery into the water in the conduit (stage 214) and/or to provide a specified pattern of illumination (stage 216).

Method 200 may further comprise configuring the UV LEDs to provide a disinfection efficiency that is higher than a disinfection efficiency of a commercial UV lamp having similar dimensions and illumination power (stage 218).

In some embodiments, method 200 may further comprise arranging the UV LEDs on the supporting structure to have an increasing number of the UV LEDs from a proximal end of the supporting structure adjacent to walls of the conduit—toward a distal end of the supporting structure away from the walls of the conduit and towards a center of the conduit (stage 220).

In some embodiments, method 200 may further comprise arranging the UV LEDs on the supporting structure in a spatial relation to at least one reflective element associate with the conduit—to maximize the efficiency of UV delivery, taking into account a position and orientation of the at least one reflective element (stage 222).

Method 200 may further comprise removing heat from the operating UV LEDs (stage 232), e.g., by enhancing heat dissipation from the densely packed UV LEDs by implementing various thermal management solutions, such as improving thermal contact to the UV LEDs by using thermal paste(s), integrating heat removal elements such as heat pipes within the illumination source and heat exchangers associated with the heat pipes, to efficiently remove heat form the operating UV LEDs to, maintain operational conditions for the UV LEDs and comply with specific limits for allowable heat dispersal within the conduit.

In some embodiments, method 200 may further comprise operating the UV LEDs in a phased control configuration of the UV LEDs to temporally adjust the illumination intensity at specified locations (stage 234). Optionally, method 200 may further comprise measuring water flow at one or multiple locations in the conduit and adjusting an illumination pattern according to the measured water flow (stage 236), possibly implementing a feedback loop to maximize the efficiency of disinfection.

Method 200 may further comprise delivering the UV illumination from the LEDs and from at least one UV lamp and arranging the UV LEDs on the supporting structure to maximize the efficiency of UV delivery, taking into account an illumination distribution of the at least one UV lamp (stage 240).

Method 200 may further comprise arranging and/or operating the UV LEDs on the supporting structure to provide an illumination intensity that increases at radial sections characterized by shorter effective illumination distances (stage 242). In various embodiments, the modification of the illumination intensity may be carried out by adjustment of the currents delivered to the UV LEDs and/or by switching the UV LEDs to operation for a duration proportional to the required illumination intensity (stage 244).

Method 200 may further comprise delivering the UV illumination from multiple supporting structures with attached UV LEDs, and configuring a spatial distribution of the UV LEDs on each of the supporting structures to maximize the efficiency of UV delivery, taking into account relative positions of the illumination sources (stage 250).

FIG. 7 is a high-level block diagram of exemplary controllers 60, which may be used with embodiments of the present invention. Controller(s) 60 may include one or more controller or processor 63 that may be or include, for example, one or more central processing unit processor(s) (CPU), one or more Graphics Processing Unit(s) (GPU or general-purpose GPU—GPGPU), a field-programmable gate array (FPGA), an application-specific integrated circuit (ASIC), a digital signal processor (DSP), a microprocessor, a chip, a microchip, an integrated circuit (IC), or any other suitable multi-purpose or specific processor, controller or computational device, an operating system 61, a memory 62, a storage 65, input devices 66 and output devices 67.

Operating system 61 may be or may include any code segment designed and/or configured to perform tasks involving coordination, scheduling, arbitration, supervising, controlling, or otherwise managing operation of controller(s) 60, for example, scheduling execution of programs. Memory 62 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 62 may be or may include a plurality of possibly different memory units. Memory 62 may store for example, instructions to carry out a method (e.g., code 64), and/or data such as user responses, interruptions, etc.

Executable code 64 may be any executable code, e.g., an application, a program, a process, task or script. Executable code 64 may be executed by controller 63 possibly under control of operating system 61. For example, executable code 64 may when executed cause the production or compilation of computer code, or application execution such as VR execution or inference, according to embodiments of the present invention. Executable code 64 may be code produced by methods described herein. For the various modules and functions described herein, one or more computing devices and/or components of controller(s) 60 may be used. Devices that include components similar or different to those included in controller(s) 60 may be used and may be connected to a network and used as a system. One or more processor(s) 63 may be configured to carry out embodiments of the present invention by for example executing software or code.

Storage 65 may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, VR model data, parameters, etc. may be stored in a storage 65 and may be loaded from storage 65 into a memory 62 where it may be processed by controller 63. In some embodiments, some of the components shown in FIG. 7 may be omitted.

Input devices 66 may be or may include for example a mouse, a keyboard, a touch screen or pad or any suitable input device. It will be recognized that any suitable number of input devices may be operatively connected to controller(s) 60 as shown by block 66. Output devices 67 may include one or more displays, speakers and/or any other suitable output devices. It will be recognized that any suitable number of output devices may be operatively connected to controller(s) 60 as shown by block 67. Any applicable input/output (I/O) devices may be connected to controller(s) 60, for example, a wired or wireless network interface card (NIC), a modem, printer or facsimile machine, a universal serial bus (USB) device or external hard drive may be included in input devices 66 and/or output devices 67.

Embodiments of the invention may include one or more article(s) (e.g., memory 62 or storage 65) such as a computer or processor non-transitory readable medium, or a computer or processor non-transitory storage medium, such as for example a memory as disclosed herein, a disk drive, or a USB flash memory, encoding, including or storing instructions, e.g., computer-executable instructions, which, when executed by a processor or controller, carry out methods disclosed herein.

Elements from FIGS. 1A-6 may be combined in any operable combination, and the illustration of certain elements in certain figures and not in others merely serves an explanatory purpose and is non-limiting.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. An illumination source for delivering ultraviolet (UV) illumination in a water treatment system, the illumination source comprising a plurality of UV LEDs (light emitting diodes) attached to a supporting structure that is insertable into the water treatment system, protruding into a conduit holding the water-, wherein the supporting structure has a shape of a commercial UV lamp and is insertable into a UV-transparent casing for housing the commercial UV lamp.

2. (canceled)

3. The illumination source of claim 1, wherein the UV LEDs are arranged on the supporting structure to maximize an efficiency of UV delivery into the water in the conduit.

4. The illumination source of claim 1, wherein the UV LEDs are arranged on the supporting structure to provide a specified pattern of illumination.

5. The illumination source of claim 1, wherein a disinfection efficiency of the illumination source is higher than a disinfection efficiency of a commercial UV lamp having similar dimensions and illumination power.

6. An illumination source for delivering ultraviolet (UV) illumination in a water treatment system, the illumination source comprising a plurality of UV LEDs (light emitting diodes) attached to a supporting structure that is insertable into the water treatment system, protruding into a conduit holding the water, wherein: a number of the UV LEDs increases from a proximal end of the supporting structure adjacent to walls of the conduit—toward a distal end of the supporting structure away from the walls of the conduit and towards a center of the conduit, and/or a number of the UV LEDs and/or an operation intensity thereof increases at radial sections characterized by shorter effective illumination distances.

7. (canceled)

8. The illumination source of claim 6, further configured to operate the UV LEDs in a phased control configuration of the UV LEDs to temporally adjust the illumination intensity at specified locations.

9. The illumination source of claim 6, wherein modification of the illumination intensity is carried out by adjustment of the currents delivered to the UV LEDs and/or by switching the UV LEDs to operation for a duration proportional to the required illumination intensity.

10. The illumination source of claim 1, wherein the water treatment system comprises at least one reflective element, and the UV LEDs are arranged to maximize the efficiency of UV delivery, taking into account a position and orientation of the at least one reflective element.

11. The illumination source of claim 1, wherein the illumination source comprises in-water wireless LEDs.

12. The illumination source of claim 1, wherein the UV LEDs are connected to modular MCPCB (metal core printed circuit board) strips attached to the supporting structure.

13. The illumination source of claim 1, further comprising heat dispersal elements configured to remove heat from the UV LEDs during operation.

14. The illumination source of claim 13, wherein the supporting structure is shaped as a sleeve that surrounds the heat dispersal elements, which comprise two opposite elements with interspaced heat conveying pipes, and associated heat removing elements.

15. A water treatment system comprising at least one UV lamp and at least one of the illumination source of claim 1, wherein a spatial distribution of the UV LEDs of the at least one illumination source is determined to maximize the efficiency of UV delivery, taking into account an illumination distribution of the at least one UV lamp.

16. A water treatment system comprising a plurality of the illumination sources of claim 1, wherein a spatial distribution of the UV LEDs on each illumination source is determined to maximize the efficiency of UV delivery, taking into account relative positions of the illumination sources.

17. The water treatment system of claim 16, further comprising at least one controller configured to modify an illumination pattern of the UV LEDs of the at least one illumination source.

18. The water treatment system of claim 17, further comprising at least one water flow sensor configured to measure water flow in the conduit and wherein the at least one controller is further configured to adjust the illumination pattern according to the measured water flow.

19. A method of delivering ultraviolet (UV) illumination for water disinfection, the method comprising:
attaching a plurality of UV LEDs (light emitting diodes) to a supporting structure that is insertable and protrudes into a water conduit holding the water, configuring the supporting structure to have a shape of a commercial UV lamp, and
operating the UV LEDs to disinfect the water.

20.-23. (canceled)

24. The method of claim 19, further comprising arranging the UV LEDs on the supporting structure to have an increasing number of the UV LEDs from a proximal end of the supporting structure adjacent to walls of the conduit—toward a distal end of the supporting structure away from the walls of the conduit and towards a center of the conduit.

25. The method of claim 19, further comprising arranging and/or operating the UV LEDs on the supporting structure to provide an illumination intensity that increases at radial sections characterized by shorter effective illumination distances.

26.-30. (canceled)

31. The method of claim 19, further comprising delivering the UV illumination from multiple supporting structures with attached UV LEDs, and configuring a spatial distribution of the UV LEDs on each of the supporting structures to maximize the efficiency of UV delivery, taking into account relative positions of the illumination sources.

32. (canceled)
